(19) United States
(12) Patent Application Publication
(10) Pub. No.: US 2026/0355394 A1
(43) Pub. Date: Nov. 12, 2026

(54) AIR BAG COMPARTMENT STRUCTURE OF BREAST PUMP

(71) Applicant: FOSHAN SHUNDE VISION ELECTRIC MFG. CO., LTD., Foshan, Guangdong (CN)

(72) Inventors: Junwu Liang, Zhongshan City, Guangdong Province (CN); Shuihua Liu, Zhongshan City, Guangdong Province (CN)

(21) Appl. No.: 18/796,556

(22) PCT Filed: Jan. 3, 2024
(86) PCT No.: PCT/CN2024/070463

§ 371 Date: Jul. 19, 2026

(30) Foreign Application Priority Data
Aug. 10, 2023 (CN) 202322148060.5

(51) Int. Cl. A61M 1/06; A61M 39/22

(52) U.S. Cl. CPC A61M 1/062; A61M 39/228

(57) ABSTRACT

The present utility model provides the air bag compartment structure of the breast pump, including a milk suction cover and an air bag assembly, where an air passage cavity is provided on a rear side of the milk suction cover, the air bag assembly is mounted in the air passage cavity, a front side end and a rear side end of the air bag assembly are hermetically connected to the air passage cavity, an air bag body is provided on the air bag assembly, a milk suction passage in bag body, a suction region is formed between an outer side of the air bag body and the air passage cavity, and a suction port in communication with the suction region is provided on the air passage cavity. The entire air bag compartment structure is reasonable in design, is composed of the milk suction cover, the air bag body and a structural frame, includes a few components, is easy to assemble, and is convenient to assemble, disassemble and clean during use by a user.

### TECHNICAL FIELD

The present utility model relates to the field of breast pumps, in particular to an air bag compartment structure of a breast pump.

### BACKGROUND

Breast pumps are devices capable of sucking breast milk from human bodies by simulating infant suction. There are many types of breast pumps, including electric breast pumps, manual breast pumps and pressing type breast pumps. An air bag compartment is an important component for forming the negative pressure in the breast pump. Since an air bag compartment part needs to be disassembled, cleaned and disinfected after the breast pump is used each time, the structural design of the air bag compartment affects later use experience of a user. However, through market research, it is found that the air bag compartments at the current stage have the problems of uneasiness in mounting, disassembly and cleaning by the user due to excessive structural components, incapability of sucking the milk due to unstable or excessively low suction due to negative pressure leakage, etc.

Therefore, it is necessary to perform further improvement.

### SUMMARY

In order to solve the above problems, the present utility model provides an air bag compartment structure of a breast pump. The entire air bag compartment structure is reasonable in design and easy to assemble, disassemble, clean and disinfect.

The present utility model provides the air bag compartment structure of the breast pump, comprising a milk suction cover and an air bag assembly, where an air passage cavity is provided on a rear side of the milk suction cover, the air bag assembly is mounted in the air passage cavity, a front side end and a rear side end of the air bag assembly are hermetically connected to the air passage cavity, an air bag body is provided on the air bag assembly, a milk suction passage in communication with the milk suction cover is formed on an inner side of the air bag body, a suction region is formed between an outer side of the air bag body and the air passage cavity, and a suction port in communication with the suction region is provided on the air passage cavity.

In one or more embodiments, the air bag assembly comprises a structural frame and the air bag body, and two ends of the air bag body are respectively sleeved on two ends of the structural frame.

In one or more embodiments, each of the two ends of the air bag body is provided with an annular sleeve portion turned outwards, each of front and rear ends of the structural frame is provided with an annular step connected to the respective annular sleeve portion in a matching manner, each of the annular sleeve portions is hermetically connected to the air passage cavity, and the air bag body comprises one or more protruding air bags in a diameter direction.

In one or more embodiments, a first convex rib capable of being extruded and deformed is provided on an end surface of each of the annular sleeve portions in contact with the air passage cavity, and the first convex ribs and the air bag body are of an integrated structure.

In one or more embodiments, a second convex rib capable of being extruded and deformed is provided on an end surface of an outer side of each of the annular sleeve portions, a limiting step is provided at a front end of the air passage cavity, and the limiting step is capable of abutting against a front end of the air bag assembly.

In one or more embodiments, the second convex ribs and the air bag body are of an integrated structure, and the air bag body is made of elastic soft rubber.

The present utility model has the beneficial effects as follows: the entire air bag compartment structure is reasonable in design, is composed of the milk suction cover, the air bag body and the structural frame, comprises a few components, is easy to assemble, and is convenient to assemble, disassemble and clean during use by a user.

### BRIEF DESCRIPTION OF THE DRAWINGS

FIG. 1 is a sectional view of an air bag compartment passing through a central axis in the present application; and

FIG. 2 is a structural schematic diagram of a milk suction cover and an air bag assembly after disassembly in the present application.

### DETAILED DESCRIPTION OF EMBODIMENTS

In order to enhance the understanding of the present utility model, the present utility model is further described in detail below in conjunction with embodiments and accompanying drawings, and the embodiments are only used for explaining the present utility model, and do not limit the scope of protection of the present utility model.

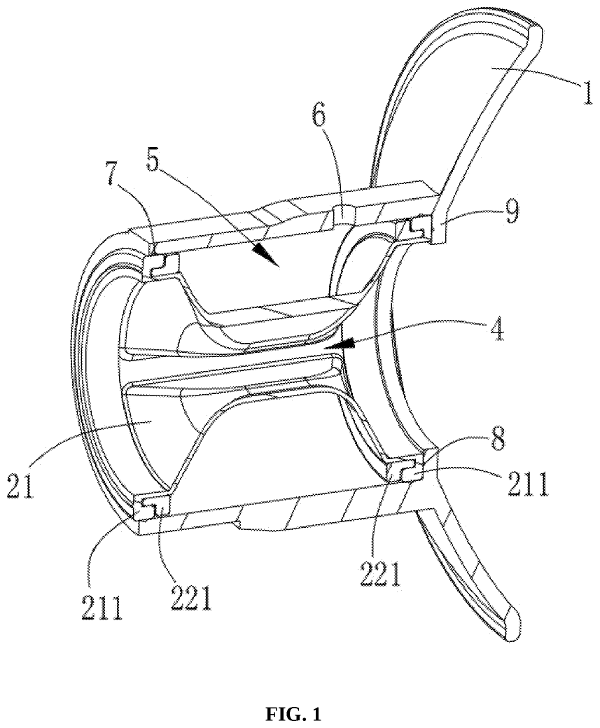

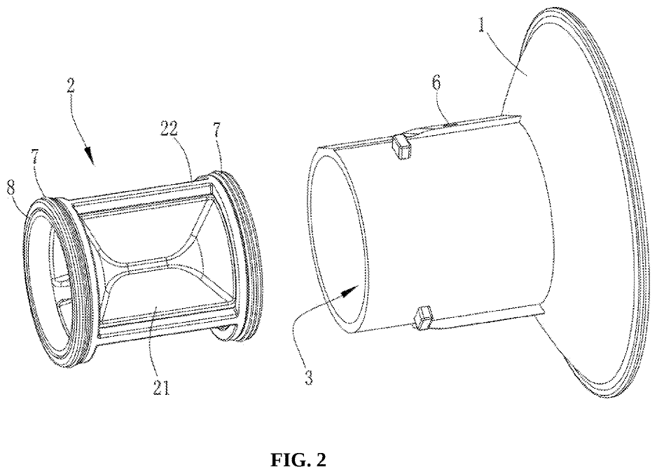

Reference is made to FIGS. 1-2. The present utility model provides an air bag compartment structure of a breast pump, including a milk suction cover 1 and an air bag assembly 2, where an air passage cavity 3 is provided on a rear side of the milk suction cover 1, the air bag assembly 2 is mounted in the air passage cavity 3, a front side end and a rear side end of the air bag assembly 2 are hermetically connected to the air passage cavity 3, an air bag body 21 is provided on the air bag assembly 2, a milk suction passage 4 in communication with the milk suction cover 1 is formed on an inner side of the air bag body 21, a suction region 5 is formed between an outer side of the air bag body 21 and the air passage cavity 3, and a suction port 6 in communication with the suction region is provided on the air passage cavity 3. In this solution, the entire air bag compartment structure is composed of the milk suction cover 1, the air bag body 21 and a structural frame 22, two ends of the air bag body 21 are sleeved on two ends of the structural frame 22, and the air bag body is mounted on the structural frame and is mounted into the air passage cavity by means of the structural frame, so that the air bag compartment structure has the advantages of simple structure and easiness in assembly and disassembly and is convenient to use and clean by a user.

Each of the two ends of the air bag body 21 is provided with an annular sleeve portion 211 turned outwards, each of front and rear ends of the structural frame 22 is provided with an annular step 221 connected to the respective annular sleeve portion 211 in a matching manner, and each of the annular sleeve portions 211 is hermetically connected to the air passage cavity 3. With the adoption of such structure, the mounting efficiency of the air bag body is higher, and the connection tightness of the air bag body and the structural frame is higher; in a process of inserting the structural frame into the air passage cavity, the annular steps are extruded and deformed by the air passage cavity so as to be closely attached to the air passage cavity to be sealed.

A first convex rib 7 capable of being extruded and deformed is provided on an end surface of each of the annular sleeve portions 211 in contact with the air passage cavity 3, and the first convex ribs 7 and the air bag body 21 are of an integrated structure. Providing the first convex ribs can further enhance the sealing effect between the annular sleeve portions and the air passage cavity, thereby improving the airtightness.

A second convex rib 8 capable of being extruded and deformed is provided on an end surface of an outer side of each of the annular sleeve portions 211, a limiting step 9 is provided at a front end of the air passage cavity 3, and the limiting step 9 is capable of abutting against a front end of the air bag assembly 2. The second convex ribs have a function consistent with that of the first convex ribs, and are used for enhancing the airtightness. The second convex ribs 8 and the air bag body 21 are of an integrated structure, the air bag body 21 is made of a food-grade elastic soft rubber material, and the structural frame is made of a hard food-grade material, so that the entire air bag assembly is supported by both elastic soft rubber and the hard structural frame, can effectively form a negative-pressure compartment, and is convenient to mount, disassemble and disinfect.

The above are only preferred embodiments of the present utility model, and the scope of protection of the present utility model is not limited by the above embodiments. However, any equivalent modification or variation made by those of ordinary skill in the art according to the content disclosed in the present utility model should be included in the scope of protection recited in the claims.

1. An air bag compartment structure of a breast pump, comprising a milk suction cover (1) and an air bag assembly (2), wherein an air passage cavity (3) is provided on a rear side of the milk suction cover (1), the air bag assembly (2) is mounted in the air passage cavity (3), a front side end and a rear side end of the air bag assembly (2) are hermetically connected to the air passage cavity (3), an air bag body (21) is provided on the air bag assembly (2), a milk suction passage (4) in communication with the milk suction cover (1) is formed on an inner side of the air bag body (21), a suction region (5) is formed between an outer side of the air bag body (21) and the air passage cavity (3), and a suction port (6) in communication with the suction region is provided on the air passage cavity (3).

2. The air bag compartment structure of a breast pump according to claim 1, wherein the air bag assembly (2) comprises a structural frame (22) and the air bag body (21), and two ends of the air bag body (21) are respectively sleeved on two ends of the structural frame (22).

3. The air bag compartment structure of a breast pump according to claim 2, wherein each of the two ends of the air bag body (21) is provided with an annular sleeve portion (211) turned outwards, each of front and rear ends of the structural frame (22) is provided with an annular step (221) connected to the respective annular sleeve portion (211) in a matching manner, each of the annular sleeve portions (211) is hermetically connected to the air passage cavity (3), and the air bag body comprises one or more protruding air bags in a diameter direction.

4. The air bag compartment structure of a breast pump according to claim 3, wherein a first convex rib (7) capable of being extruded and deformed is provided on an end surface of each of the annular sleeve portions (211) in contact with the air passage cavity (3), and the first convex ribs (7) and the air bag body (21) are of an integrated structure.

5. The air bag compartment structure of a breast pump according to claim 3 or 4, wherein a second convex rib (8) capable of being extruded and deformed is provided on an end surface of an outer side of each of the annular sleeve portions (211), a limiting step (9) is provided at a front end of the air passage cavity (3), and the limiting step (9) is capable of abutting against a front end of the air bag assembly (2).

6. The air bag compartment structure of a breast pump according to claim 5, wherein the second convex ribs (8) and the air bag body (21) are of an integrated structure, and the air bag body (21) is made of elastic soft rubber.
